(19) United States
(12) Patent Application Publication
(10) Pub. No.: US 2025/0042741 A1
(43) Pub. Date: Feb. 6, 2025

(54) PROCESS FOR PRODUCING ALKALI SALTS OF BIS(FLUOROSULFONYL)IMIDE

(71) Applicant: SPECIALTY OPERATIONS FRANCE, Lyon (FR)

(72) Inventors: Olivier Buisine, Saint Genis-Laval (FR); Woo-Jeong Jang, Seongdong-gu, Seoul (KR); Young-Su Kim, Seo-gu, Incheon (KR); Etienne Schmitt, Soucieu-En-Jarrest (FR)

(73) Assignee: SPECIALTY OPERATIONS FRANCE, Lyon (FR)

(21) Appl. No.: 18/722,467

(22) PCT Filed: Dec. 19, 2022
(86) PCT No.: PCT/EP2022/086648

§ 371 Date: Jun. 20, 2024

(30) Foreign Application Priority Data
Dec. 20, 2021 (EP) 21306857.0

(51) Int. Cl. C01B 21/086; C07C 29/70; H01M 10/0568

(52) U.S. Cl. CPC C01B 21/086; C07C 29/705; H01M 10/0568; C01P 2006/40

(57) ABSTRACT

The invention relates to a process for manufacturing bis(fluoro sulfonyl)imide salt(s) (MFSI) comprising at least a step of reacting an ammonium salt of bis(fluorosulfonyl)imide (NH₄FSI) with a fluorine-containing alkoxide. The invention also relates to a process for preparing the fluorine-containing alkoxide.

### CROSS-REFERENCE TO RELATED PATENT APPLICATIONS

This application claims priority filed on 20 Dec. 2021 in EUROPE with Nr 21306857.0, the whole content of this application being incorporated herein by reference for all purposes.

### TECHNICAL FIELD

The invention relates to a process for manufacturing bis(fluorosulfonyl)imide salt(s) (MFSI) comprising at least a step of reacting an ammonium salt of bis(fluorosulfonyl)imide (NH₄FSI) with a fluorine-containing alkoxide. The invention also relates to a process for preparing such a fluorine-containing alkoxide.

### BACKGROUND ART

Bis(fluorosulfonyl)imide and salts thereof, in particular the lithium salt of bis(fluorosulfonyl)imide (LiFSI), are useful compounds in a variety of technical fields. Bis(fluorosulfonyl)imide and their salts are especially useful in battery electrolytes. For the use in batteries, it is very important to limit the presence of impurities.

The production of bis(fluorosulfonyl)imide and salts thereof is described in the literature. Among the various technologies described, the majority uses a fluorination reaction with a fluorinating agent in a solvent. The subsequent step is usually a cation exchange reaction, more precisely a lithiation step when LiFSI is to be prepared.

Notably, WO 2017/090877 (CLS) describes a method for producing lithium bis(fluorosulfonyl)imide comprising the steps of: (1) reacting bis(chlorosulfonyl)imide with a fluorinating reagent in a solvent, followed by treatment with an alkaline reagent, thereby producing ammonium bis(fluorosulfonyl)imide; and (2) reacting the ammonium bis(fluorosulfonyl)imide with a lithium base. The lithiation is performed with lithium hydroxide hydrate (LiOH·H₂O) as a lithium base, in butyl acetate as a solvent. After completion of the reaction, the aqueous layer is separated and lithium bis(fluorosulfonyl)imide is produced through concentration, recrystallization and separation steps. This protocol for removing water suffers a low productivity as it is time consuming and requires fresh solvent.

WO 2020/099527 (Solvay SA) discloses a method for producing an alkali salt of bis(fluoro sulfonyl)imide, comprising the steps of: a) reacting bis(chloro sulfonyl)imide or salts thereof with ammonium fluoride to produce ammonium salt of bis(fluoro sulfonyl)imide; b) crystallizing by adding at least one precipitation solvent and separating the ammonium salt of bis(fluoro sulfonyl)imide; and c) reacting the crystallized ammonium salt of bis(fluoro sulfonyl)imide with an alkali salt to obtain alkali salt of bis(fluorosulfonyl)imide. In example 3 the alkali salt is an aqueous solution of LiOH·H₂O. No disclosure of a fluorine-containing hydroxide is made in this document.

### BRIEF DESCRIPTION OF THE INVENTION

An object of the present invention is to provide a process for manufacturing a MFSI salt, wherein M indicates a metal, and the MFSI salt is preferably LiFSI, which is less time consuming and do not require additional solvent(s).

Also, an object of the present invention is to provide a process for manufacturing a MFSI salt that does generate water as the process proceeds.

Advantageously, the process of the present invention is based on the use of an alkoxide of formula RO⁻M⁺ or of its solvate form [RO⁻M⁺][n ROH] wherein R is a moiety containing fluorine atoms.

The present invention is directed to a process for manufacturing a bis(fluoro sulfonyl)imide salt (MFSI) of formula (I):

[F—SO₂—N⁻—SO₂—F] M⁺ (I)

wherein M is selected from the group consisting of Li, Na, K, Rb, Cs and Fr.

This process is based on the use of a fluorine-containing alkoxide of formula RO⁻M⁺ or of its solvate form [RO⁻M⁺][n ROH], which effectively replace the usual alkalinization agents used until now to prepare alkali salts of bis(fluoro sulfonyl)imide. The use of such fluorine-containing alkoxide allows to advantageously avoid the generation of water in the reaction medium after the reaction and, consequently, to avoid time-consuming additional steps to remove such water.

The present invention is also directed to a process for preparing the fluorine-containing alkoxide used in the above process, and to the fluorine-containing alkoxide thus obtained.

The present invention is also directed to the use of such fluorine-containing alkoxide to prepare the bis(fluorosulfonyl)imide salt(s) (MFSI).

### DESCRIPTION OF THE INVENTION

A first aspect of the present invention relates to a process for manufacturing a bis(fluorosulfonyl)imide salt (MFSI) of formula (I):

[F—SO₂—N⁻—SO₂—F] M⁺ (I)

wherein

M is selected from the group consisting of Li, Na, K, Rb, Cs and Fr, and said process comprises reacting an ammonium salt of bis(fluoro sulfonyl)imide of formula (II) [NH₄FSI salt]:

[F—SO₂—N⁻—SO₂—F] NH₄⁺ (II)

with a fluorine-containing alkoxide of formula (III):

RO⁻M⁺ (III)

wherein
R is selected from the group consisting of C₁-C₂₀ fluoroalkyl, C₁-C₂₀ fluoroalkenyl and C₁-C₂₀ fluoroalkynyl,.
M is selected from the group consisting of Li, Na, K, Rb, Cs and Fr; and
NH₄FSI salt is optionally in a form of a solvate comprising:
   50 to 99.9 wt. %, of the NH₄FSI salt, and
   0.1 to 50 wt. %, of at least one solvent S₂, which is selected from the group consisting of cyclic and acyclic ethers.

The fluorine-containing alkoxide of formula (III) can advantagesouly be in the form of a solvate, as follows:

[RO⁻M⁺][n ROH] (III*)

n is an integer from 1 to 10 and

M and each of R has the same meanings described above.

The fluorine-containing alkoxide of formula (III) or (III*) is advantageously used to alkalinize the NH₄FSI salt. In other words, the fluorine-containing alkoxide of formula (III) or (III*) is able to transfer its alkali metal ‘M’ to NH₄FSI salt.

The fluorine-containing alkoxide (III) or (III*) may be provided in the process described herein in the solid state, in the liquid state, or in organic solution.

The fluorine-containing alkoxide of formula (III) or (III*) complies with formula RO⁻M⁺, in which R is selected from the group consisting of C₁-C₂₀ fluoroalkyl, C₁-C₂₀ fluoroalkenyl and C₁-C₂₀ fluoroalkynyl, in other words fluoralkyl, fluoroalkenyl and fluoroalkynyl groups having from 1 to 20 carbon atoms. Preferably, the number of carbon atoms within the R group ranges from 1 to 12, more preferably from 1 to 8 or from 1 to 6 and even more preferably from 1 to 3, this number being equal to 2 in a particular embodiment. Preferably, R is a fluoroalkyl group.

In some embodiments, R complies with formula R_{F}-L, wherein R_{F} is a monovalent group selected from the group consisting of C₁-C₃ perfluoroalkyl, C₁-C₃ perfluoroalkenyl and C₁-C₃ perfluoroalkynyl and L is a divalent group selected from the group consisting of C₁-C₃ alkylene, C₁-C₃ alkenylene and C₁-C₃ alkynylene; preferably R_{F} is a C₁-C₂ perfluoroalkyl group and L is a C₁-C₂ alkylene group; more preferably R_{F} is CF₃ and L is CH₂, in other words the alcohol of formula (I) is 2,2,2-trifluoroethanol (TFE). The fluorine-containing alkoxide of formula (III) or (III*) above may be prepared by a process described in detail below.

The NH₄FSI (II) involved in the process of the present invention may be in the form of a substantially pure salt or it may be in the form of a solvate, as described below. It can be prepared by any method known by the skilled person. It may be prepared by fluorinating bis(chlorosulfonyl)imide (HCSI), or salts thereof, with a fluorinating agent, for example ammonium fluoride.

In some embodiments, the NH₄FSI salt is a solvate [NH₄FSI solvate], possibly in a crystallized form, comprising:
50 to 98 wt. %, of the NH₄FSI salt, and
2 to 50 wt. %, of at least one solvent S₂, which is selected from the group consisting of cyclic and acyclic ethers.

Preferably, the NH₄FSI solvate comprises from 51 to 98 wt. %, more preferably from 55 to 95 wt. %, or from 78 to 83 wt. % of the NH₄FSI salt.

Preferably, the NH₄FSI solvate comprises from 2 to 49 wt. %, more preferably from 5 to 45 wt. % or from 17 to 22 wt. % of at least one solvent S₂.

The at least one solvent S₂ is preferably selected from the group consisting of diethylether, diisopropylether, methyl-t-butylether, dimethoxymethane, 1,2-dimethoxyethane, tetrahydrofuran, 2-methyltetrahydrofuran, dioxolane, 1,3-dioxane, 4-methyl-1,3-dioxane, and 1,4-dioxane, and mixtures thereof; more preferably from the list consisting of diethyl ether, diisopropyl ether, methyl t-butyl ether, 1,2-dimethoxyethane, tetrahydrofuran, 2-methyltetrahydrofuran, dioxane and mixtures thereof; even more preferably being 1,3-dioxane or 1,4-dioxane.

In some embodiments, the process for manufacturing the MFSI salt according to the present invention comprises a preliminary step i) of preparing NH₄FSI solvate by the following steps:
i₁) providing a crude salt of NH₄FSI;
i₂) dissolving the crude salt of NH₄FSI in at least one solvent S₁;
i₃) crystallizing the crude salt of NH₄FSI by means of at least one solvent S₂; and
i₄) separating the NH₄FSI salt from at least part of the solvents S₁ and S₂, preferably by filtration.

The crude salt of NH₄FSI may comprise 80 to 97 wt. % of the salt of NH₄FSI, preferably 85 to 95 wt. %, more preferably 90 to 95 wt. %.

The solvent S₁ is preferably selected from the group consisting of acetonitrile, valeronitrile, adiponitrile, benzonitrile, methanol, ethanol, 1-propanol, 2-propanol, 2,2,2,-trifluoroethanol, n-butyl acetate, isopropyl acetate, and mixtures thereof; preferably 2,2,2,-trifluoroethanol.

In some preferred embodiments, step i₄) consists in separating the NH₄FSI salt from:
more than 99.9 wt. % of the solvent S₁; and
50 to 99 wt. % of at least one solvent S₂.

The reaction between the NH₄FSI salt and the fluorine-containing alkoxide of formula (III) or (III*) may be carried out in one or more than one solvent(s). However, the reaction can be solvent-free.

The fluorine-containing alkoxide of formula (III) or (III*) may be provided in a state in which it is already dissolved in a solvent. In this case, the addition of solvent is possible, but not necessarily required. This additional solvent may be identical or different from the solvent(s) used in step i).

The fluorine-containing alkoxide of formula (III) or (III*) may be provided in a substantially dry solid state and be dissolved in a solvent before use in the process of the present invention.

In some embodiments, a solvent is used (added) to carry out the reaction between NH₄FSI and the fluorine-containing alkoxide (III) or (III*). It is preferred to choose the same solvent than the one used to prepare the alkoxide (III) or (III*). Alternatively, a distinct solvent may be used.

The solvent may advantageously be according to formula ROH, in which R is selected from the group consisting of C₁-C₂₀ fluoroalkyl, C₁-C₂₀ fluoroalkenyl and C₁-C₂₀ fluoroalkynyl. R is preferably selected from the group consisting of fluoroalkyl, fluoroalkenyl and fluoroalkynyl groups having from 1 to 12 carbon atoms, more preferably from 1 to 6 carbon atoms and even more preferably from 1 to 3 carbon atoms. R has 2 carbon atoms in a particular embodiment. Preferably, R is a fluoroalkyl group. According to one embodiment, R complies with formula R_{F}-L wherein R_{F} is a monovalent group selected from the group consisting of C₁-C₃ perfluoroalkyl, C₁-C₃ perfluoroalkenyl and C₁-C₃ perfluoroalkynyl and L is a divalent group selected from the group consisting of C₁-C₃ alkylene, C₁-C₃ alkenylene and C₁-C₃ alkynylene; preferably R_{F} is a C₁-C₂ perfluoroalkyl group and L is a C₁-C₂ alkylene group; more preferably R_{F} is CF₃ and L is CH₂: in other words, the alcohol of formula (I) used in the process for producing a salt of bis(fluorosulfonyl)imide may be 2,2,2-trifluoroethanol.

Such solvent may preferably be selected among solvents S₁ or S₂.

The reactants, optionally the solvent, may be contacted in any order. In particular, the solvent may be added prior to, after or at the same time as the reactants. NH₄FSI (II) may for example be dissolved in the solvent before adding the alkoxide (III) or (III*). The molar ratio solvent/NH₄FSI may range from 1 to 10, in particular from 1 to 5, more particularly from 1 to 2.

In some embodiments, the process of the present invention may be a solvent-free process. In other words, no solvent/diluent, alternatively a very low amount of solvent/diluent, is added to the reaction mixture during the reaction. The process may take place in molten NH₄FSI salt in the absence of solvent or in the presence of a solvent less than 5 wt. % based on the total weight of the reaction mixture involved in the process. According to these embodiments, the process is performed in the melt in the absence of solvents and diluents. More precisely, the molten NH₄FSI (II) may act to disperse the reactants and allow the reactants involved in the reaction to meet and react.

When the process of the invention is solvent-free, the addition of the alkoxide (III) or (III*) in the molten NH₄FSI salt may be performed sequentially, progressively or continuously. Batch reactor, extruder and mixing kneader can for example be used in the present invention. Anti-acidic corrosion material (e.g. PTFE, PFA, etc) can be coated (in other words, lined) inside the chosen reactor. Reference can be made to industrialized melt mixers or melt blenders.

The process of the present invention is preferably carried out under inert atmosphere to avoid moisture contamination. The process of the present invention may for example be carried out under nitrogen or argon.

The molar ratio alkoxide (III) or (III*)/NH₄FSI salt may range from 1 to 10, in particular from 1 to 5 and more particularly from 1 to 2.

The process of the present invention may be carried out at a temperature of less than 100° C., for example between 0° C. and 50° C., more preferably between 15° C. and 35° C., and even more preferably at about room temperature.

Preferably, the process of the present invention is carried out under atmospheric pressure, but it is not excluded to work below or above atmospheric pressure, for instance between 5 mbar and 1.5 bar, preferably between 5 mbar and 100 mbar.

The reaction time of the process of the present invention can be selected freely depending for example on the reactor used, the reaction temperature and the reactant quantities involved. It is preferable that the reaction time is from 1 to 12 hours, particularly from 1.5 to 10 hours or from 2 to 9 hours.

The MFSI salt is obtained in the reaction medium at the end of the reaction between NH₄FSI salt and the fluorine-containing alkoxide (III) or (III*).

Further steps may be carried out to purify/isolate the MFSI salt.

According to one particular embodiment, the process of the present invention comprises at least one further step of concentrating the MFSI salt, being preferably performed under reduced pressure.

This step may be performed by decreasing the temperature, by decreasing the pressure, or both. The temperature may particularly be decreased down to a temperature ranging from −10° C. to 10° C., preferably from −5° C. to 5° C., being preferably about 0° C.

The pressure may be adjusted depending on the nature of the species present in the reaction medium at the end of the reaction; it may be in particular adjusted at a value comprised between 10-2 mbar and atmospheric pressure, preferably between 1 mbar and 500 mbar, preferably between 5 mbar and 100 mbar and more preferably between 10 and 30 mbar.

The operation may be repeated one or several times to purify further the salt. Fresh solvent may be added to the concentrated reaction medium containing the salt prior to the subsequent concentration operation.

A mixture of the purified alkali salt of bis(fluorosulfonyl)imide within said solvent can be obtained thereby.

Further treatments may be carried out in order to recover very pure alkali salt of bis(fluorosulfonyl)imide. Additional steps may comprise filtration, extraction, recrystallization, purification by chromatography, drying and/or formulation.

All raw materials used in the process described herein, including solvents and reagents, preferably show very high purity criteria. Preferably, their content of metal components such as Na, K, Ca, Mg, Fe, Cu, Cr, Ni, Zn, is below 10 ppm, more preferably below 2 ppm.

In addition, some of the steps or all steps of the process according to the invention are advantageously carried out in equipment capable of withstanding corrosion. For this purpose, materials intended to be in contact with the reaction medium are selected from corrosion-resistant materials, such as the alloys based on molybdenum, chromium, cobalt, iron, copper, manganese, titanium, zirconium, aluminum, carbon and tungsten, sold under the Hastelloy® brands or the alloys of nickel, chromium, iron and manganese to which copper and/or molybdenum are added, sold under the name Inconel® or Monel™, and more particularly the Hastelloy C276 or Inconel 600, 625 or 718 alloys. Stainless steels may also be selected, such as austenitic steels and more particularly the 304, 304 L, 316 or 316 L stainless steels. A steel having a nickel content of at most 22% by weight, preferably of between 6% and 20% and more preferentially of between 8% and 14%, is used. The 304 and 304L steels have a nickel content that varies between 8% and 12%, and the 316 and 316L steels have a nickel content that varies between 10% and 14%. More particularly, 316L steels are chosen. Use may also be made of equipment consisting of or coated with a polymeric compound resistant to the corrosion by the reaction medium. Mention may in particular be made of materials such as PTFE (polytetrafluoroethylene or Teflon) or PFA (perfluoroalkyl resins). Glass equipment may also be used. It will not be outside the scope of the invention to use an equivalent material. As other materials capable of being suitable for being in contact with the reaction medium, mention may also be made of graphite derivatives. Materials for filtration have to be compatible with the medium used. Fluorinated polymers (PTFE, PFA), loaded fluorinated polymers (Viton™), as well as polyesters (PET), polyurethanes, polypropylene, polyethylene, cotton, and other compatible materials can be used.

A second aspect of the present invention relates to the metal salt of bis(fluorosulfonyl)imide (MFSI) obtainable by the process of the present invention. The MFSI salt advantageously shows at least one of the following features (preferably all):
a purity of at least 98 wt. %, for example between 99 wt. % and 100 wt. % or between 99.50 and 100%, as determined by ¹⁹F NMR,
a solvent content of less than 20 wt. %, less than 10 wt. %, less than 1 wt. %, preferably between 0 wt. % and 1 wt. %, as determined by GC (alternatively headspace GC),
a moisture content of less than 500 ppm, less than 100 ppm, less than 50 ppm or even less than 20 ppm, as determined by Karl Fisher water titration, for example performed in a glovebox.

The MFSI salt of the present invention advantageously shows at least one of the following features (preferably all):
a chloride (Cl—-) content of below 100 ppm, preferably below 50 ppm, more preferably below 10 ppm, or more preferably below 2 ppm;
a fluoride (F—) content of below 100 ppm, preferably below 50 ppm, more preferably below 40 ppm, more preferably below 30 ppm, more preferably below 20 ppm; and
a sulfate (SO₄²⁻) content of below 100 ppm, preferably below 50 ppm, more preferably below 10 ppm, or more preferably below 2 ppm.

Fluoride and chloride contents may for example be measured by titration by argentometry using ion selective electrodes (or ISE). Sulfate content may alternatively be measured by ionic chromatography or by turbidimetry.

Preferably, the MFSI salt of the present invention presents at least one of the following contents of metal elements (preferably all):
an iron (Fe) content of below 100 ppm, preferably below 50 ppm, more preferably below 10 ppm;
a chromium (Cr) content of below 100 ppm, preferably below 50 ppm, more preferably below 10 ppm;
a nickel (Ni) content of below 100 ppm, preferably below 50 ppm, more preferably below 10 ppm;
a zinc (Zn) content of below 100 ppm, preferably below 50 ppm, more preferably below 10 ppm;
a copper (Cu) content of below below 100 ppm, preferably below 50 ppm, more preferably below 10 ppm;
a copper (Cu) content of below below 100 ppm, preferably below 50 ppm, more preferably below 10 ppm;
a manganese (Mg) content of below 100 ppm, preferably below 50 ppm, more preferably below 10 ppm;
a sodium (Na) content of below 100 ppm, preferably below 50 ppm, more preferably below 10 ppm;
a potassium (K) content of below 100 ppm, preferably below 50 ppm, more preferably below 10 ppm;
a (Pb) content of below 100 ppm, preferably below 50 ppm, more preferably below 10 ppm.

Elemental impurity content may for example be measured by ICP-AES (inductively coupled plasma); more specifically, Na content can be measured by AAS (atomic absorption spectroscopy).

In some embodiments, the MFSI salt of the present invention is a lithium salt of bis(fluorosulfonyl)imide, Li⁺ (FSO₂)₂N⁻(LIFSI). The lithium salt of bis(fluorosulfonyl)imide may be characterized by the following impurity profile:
a purity of at least 99.90 wt. % and varying between 99.90 wt. % and 100 wt. %, as determined by ¹⁹F NMR, and
a moisture content of less than 50 ppm, as determined by Karl Fischer water titration.

A third aspect of the present invention relates to the use of the fluorine-containing alkoxide of formula (III) or (III*):

RO⁻M⁺ (III)

[RO⁻M⁺][n ROH] (III*)

wherein.
each R is independently selected from the group consisting of C₁-C₂₀ fluoroalkyl, C₁-C₂₀ fluoroalkenyl and C₁-C₂₀ fluoroalkynyl, and
M is an alkali metal, preferably selected from the group consisting of Li, Na, K, Rb, Cs and Fr, and
n is an integer ranging from 1 to 10,
   to manufacture a metal salt of bis(fluorosulfonyl)imide (MFSI) of formula (I):

[F—SO₂—N⁻—SO₂—F] M⁺ (I).

The fluorine-containing alkoxide (III) or (III*) used in the process of the present invention to manufacture a metal salt of MFSI, contains a R-moeity, which is selected from the group consisting of C₁-C₂₀ fluoroalkyl, C₁-C₂₀ fluoroalkenyl and C₁-C₂₀ fluoroalkynyl, in other words fluoralkyl, fluoroalkenyl and fluoroalkynyl groups having from 1 to 20 carbon atoms. Preferably, the number of carbon atoms within the R group ranges from 1 to 12, more preferably from 1 to 8 or from 1 to 6 and even more preferably from 1 to 3, this number being equal to 2 in a particular embodiment. Preferably, R is a fluoroalkyl group.

In some embodiments, R complies with formula R_{F}-L wherein R_{F} is a monovalent group selected from the group consisting of C₁-C₃ perfluoroalkyl, C₁-C₃ perfluoroalkenyl and C₁-C₃ perfluoroalkynyl and L is a divalent group selected from the group consisting of C₁-C₃ alkylene, C₁-C₃ alkenylene and C₁-C₃ alkynylene; preferably R_{F} is a C₁-C₂ perfluoroalkyl group and L is a C₁-C₂ alkylene group; more preferably R_{F} is CF₃ and L is CH₂, in other words the alcohol of formula (I) is 2,2,2-trifluoroethanol (TFE).

A fourth aspect of the invention relates to a process for preparing the fluorine-containing alkoxide (III) or (III*) used in the process of the present invention, which comprises the step of reacting an alcohol of formula (IV):

ROH (IV)

with an alkali hydroxide of formula (V), or an hydrate thereof:

MOH (V)

wherein R and M are as described above.

Preferably, M represents an alkali metal selected from the group consisting of Li, Na, K, Rb, Cs and Fr.

“Hydrate” is intended to indicate any compound of formula (V) containing water in the form of H₂O molecules, usually, but not always, with a definite content of water by weight.

Preferably, the metal M is selected from the group consisting of Li, Na, K and Cs; more preferably M is Li.

The metal hydroxide of formula (V) is preferably lithium hydroxide. In particular, in one embodiment, the metal hydroxide of formula (V) used in the process for producing the flurorine-containing alkoxide used in the process of the present invention is LiOH·H₂O.

The fluorine-containing alkoxide (III) of the present invention can be in the form of a solvate as represented in formula (III*) above, that is to say a crystalline solid that contain molecules of solvent inside the crystal assembly. Crystal solvates may preferably be formed a crystallization process with the help of a solvent.

The alcohol of formula (IV) and the metal hydroxide of formula (V) or the hydrate thereof, can be contacted in various manners. They are preferably contacted under inert atmosphere, typically nitrogen or argon atmosphere. The molar ratio alcohol/metal hydroxide preferably ranges from 1 to 30, preferably from 1 to 20, more preferably from 2 to 10, even more preferably from 3 to 6. It is advantageous to solubilize the metal hydroxide (V) into the alcohol (IV), in order for the alcohol to act as a solvent, in addition to its role as a reactant. The metal hydroxide (V) is preferably at a high concentration in the alcohol (IV). To ease the dissolution of the metal hydroxide (V) in the alcohol (IV), the reaction medium may be stirred for a sufficient period of time, which may be at least 30 min, for example for 30 min to 6 hours, in particular for 30 min to 3 hours, for example during about 1 hour. The reaction can be carried out at a temperature of at least 5° C., for example ranging from 5 to 200° C., preferably from 10 to 100° C., more preferably from 15 to 50° C., even more preferably from 15 to 30° C. The reaction can preferably be carried out at ambient temperature, which is economically advantageous. Preferably, the reaction is carried out under atmospheric pressure, but it is not excluded to work below or above atmospheric pressure, for instance between 5 mbar and 1.5 bar, preferably between 5 mbar and 100 mbar.

The fluorine-containing alkoxide (III) or (III*) described herein, as obtained from the process described above, may be used as such in the process for manufacturing a bis(fluorosulfonyl)imide salt (MFSI) (I) described above. The fluorine-containing alkoxide described herein, may also be provided in a solid form and/or purified form, in order to ease its storage and implementation into the process.

In some embodiments, the process for producing the fluorine-containing alkoxide (III) or (III*) comprises a further step of concentrating the fluorine-containing alkoxide (III) or (III*), for example by evaporating part of the alcohol of formula (IV). This concentration may be carried out by heating the reaction mixture and/or by decreasing the pressure. According to one embodiment, the concentration step may consists in a distillation of the alcohol of formula (I) at a temperature comprised between 0° C. and 120° C., preferably between 5° C. and 80° C., more preferably between 10° C. and 70° C. The pressure may be adjusted depending on the nature of the alcohol of formula (I), typically between atmospheric pressure and 10-2 mbar, preferably between 1 mbar and 500 mbar, and more preferably between 5 mbar and 100 mbar. The distillation may be performed by any typical means known by the person skilled in the art on a continuous process mode or on a discontinuous/batch mode, for example a continuous batch mode solvent evaporation, a batch distillation, a continuous flow distillation of a short path, or a thin film evaporator.

In some embodiments, the process for producing the fluorine-containing alkoxide (III) or (III*) comprises a further step of crystallization and separation. The crystallization may be performed by any suitable method available to the skilled person, such as the evaporation of the remaining alcohol (IV), the addition of an additional solvent, distinct from the alcohol (IV), also called antisolvent or drown-out, the solvent layering or the sublimation. In a preferred embodiment, the crystallization is carried out by decreasing the temperature of the reaction mixture to a temperature at which crystals (i.e. crystalline solids) form. In other words, the temperature may be decreased to a value below the temperature of solubility of the alkoxide (III) or (III*). Preferably, the temperature of the reaction mixture is decreased to a value comprised between the boiling point of the alcohol of formula (IV) and −20° C., more preferably between 70° C. and −10° C., and even more preferably between 30° C. and 0° C. During the decreasing of the temperature, the pressure is preferably kept constant. However, it is not excluded to reduce the pressure simultaneously. It may cause the evaporation of part of the alcohol of formula (IV) from the reaction medium. The pressure may be decreased to a value comprised between 10⁻² mbar and atmospheric pressure (1013.25 mbar), preferably between 1 mbar and 500 mbar, and more preferably between 5 mbar and 100 mbar. The decreased temperature may be maintained for a time ranging from 1 to 20 hours, in particular from 2 to 15 hours, more particularly for 3 to 10 hours.

The separation of the fluorine-containing alkoxide (III) or (III*) may be performed by any typical separation method available to the person skilled in the art, for example by filtration. Filtration may be carried out at atmospheric pressure, under pressure or under vacuum. Mesh size of the filtration medium may be of 2 μm or below, more preferably of 0.45 μm or below, and even more preferably of 0.22 μm or below. Separated product may be washed once or several times with appropriate solvent, such as any solvent where the fluorine-containing alkoxide (III) or (III*) is insoluble and where the alcohol of formula (IV) is at least partly soluble. In addition the selected solvent should be easily separated from the alcohol of formula (IV) by any means know from the skilled person, like distillation or phase separation. The selected solvent should preferably form no azeotrope with the alcohol of formula (IV).

The crystallization and separation steps may be carried out once or may be repeated twice or more if necessary to improve the purity of the separated fluorine-containing alkoxide (III) or (III*). When repeating the procedure, the alkoxide (III) or (III*) may be solubilized in fresh alcohol of formula (IV), being preferably identical to the one used in the first occurrence. The same conditions as the ones explained above may be applied (molar ratio alcohol/alkali hydroxide, time, temperature, pressure, etc.).

In some embodiments, the process for producing the fluorine-containing alkoxide (III) or (III*) comprises a further step, after the crystallization and separation steps described above, consisting in drying the alkoxide (III) or (III*). A substantially dry solid product may be obtained from such additional steps, with all the advantages of having a product which is easier to store and use in a reaction for producing a salt of (bisfluorosulfonyl)imide, in particular the one according to the invention. The drying may be carried out by any methods available to the person skilled in the art, typically under reduced pressure and/or by heating and/or with an inert gas flow, typically a nitrogen or argon flow.

According to a preferred embodiment, the drying of the alkoxide (III) or (III*) is carried out under reduced pressure. The pressure may in particular be decreased to a value comprised between 10⁻² mbar and atmospheric pressure (1013.25 mbar), preferably between 10⁻¹ mbar and 500 mbar, preferably between 1 mbar and 100 mbar, and more preferably between 1 mbar and 10 mbar. Temperature may be comprised between 5 and 50° C., preferably between 10 and 30° C. Advantageously the drying of the alkalinization agent can be performed at room temperature. The drying time may range from 1 to 20 hours, preferably from 1 to 10 hours.

The fluorine-containing alkoxide (III) or (III*) is obtained at the end of the process described above.

A fifth aspect of the present invention relates to a fluorine-containing alkoxide of formula (III) or (III*):

RO⁻M⁺ (III)

[RO⁻M⁺][n ROH] (III*)

wherein.
each R is independently selected from the group consisting of C₁-C₂₀ fluoroalkyl, C₁-C₂₀ fluoroalkenyl and C₁-C₂₀ fluoroalkynyl,
M represents a metal, preferably selected from the group consisting of Li, Na, K, Rb, Cs and Fr, and
n is an integer ranging from 1 to 10, preferably between 1 and 5, in particular from 1 to 3, especially it can be equal to 2.

The fluorine-containing alkoxide represented in formula (III*) is in the form of a solvate, that-is-to-say crystalline solids that contain the molecules of solvent inside their crystal assembly (stoichiometrically or non-stoichiometrically), as characterized by fluorine nuclear magnetic resonance (NMR) analysis.

Preferably in formulae (III) and (III*), each R is selected from fluoroalkyl, fluoroalkenyl and fluoroalkynyl groups having from 1 to 12 carbon atoms, more preferably from 1 to 6 carbon atoms, more preferably from 1 to 3 carbon atoms, especially 2 carbon atoms. Preferably, R is a fluoroalkyl group. According to one embodiment, R complies with formula R_{F}-L wherein R_{F} is a monovalent group selected from the group consisting of C₁-C₃ perfluoroalkyl, C₁-C₃ perfluoroalkenyl and C₁-C₃ perfluoroalkynyl and L is a divalent group selected from the group consisting of C₁-C₃ alkylene, C₁-C₃ alkenylene and C₁-C₃ alkynylene; preferably R_{F} is a C₁-c₂ perfluoroalkyl group and L is a C₁-C₂ alkylene group; more preferably R_{F} is CF₃ and L is CH₂.

Preferably in each of formulae (III) and (III*), M represents an alkali metal selected from the group consisting of Li, Na, K and Cs; more preferably M is Li.

According to one specific embodiment, in the compound of formula (III*), R is CF₃-CH₂, M is Li and n=2, in other words a solvate of lithium 2,2,2-trifluoroethoxide and 2,2,2-trifluoroethanol. Preferably, this compound is a crystalline solid.

Advantageously, the alkoxide (III) or (III*) obtained by the process described in the present invention has a very high purity notably a purity of at least 98 wt. %, for example between 99 wt. % and 100 wt. % or between 99.50 and 100%, as determined by ¹⁹F NMR.

The MFSI salt, notably the LiFSI salt, obtainable by the process according to the invention, may be advantageously used in electrolyte compositions for batteries.

Should the disclosure of any patents, patent applications, and publications which are incorporated herein by reference conflict with the description of the present application to the extent that it may render a term unclear, the present description shall take precedence.

The invention will now be further described in examples without intending to limit it.

### EXAMPLES

### Example 1

Synthesis of an Alkoxide of Formula [CF₃CH₂O—Li⁺] [2 CF₃CH₂OH]

Under nitrogen, 7.7 g (0.18 mol) of LiOH monohydrate was mixed with 143 g (1.43 mol) of 2,2,2-trifluoroethanol (TFE) and stirred at room temperature for 1 hour. The mixture was cooled to T=5° C. for 2 hours. Some crystals were observed in a cloudy suspension. The crystals were isolated by filtration. The crystals were then dissolved in 145 g (1.45 mol) of fresh TFE at room temperature for 1 hour and cooled to T=5° C. for 2 hours. A solid was obtained by filtration. It was then dried under vacuum (P=5 mbar) for 5 hours at room temperature. NMR analysis is consistent with a solvate compound of formula [CF₃CH₂O—Li⁺] [2 CF₃CH₂OH].

### Example 2

Synthesis of Lithium Salt of bis(fluorosulfonyl)imide Using the Solvate Alkoxide of Example 1

Under nitrogen atmosphere, a solution of 6.9 g (0.35 mmol) ammonium bis(fluorosulfonyl)imide (NH₄FSI) was prepared in 60 g (0.60 mol) of TFE. 10.6 g (0.035 mmol) of the solid alkoxide of example 1 was then added to the vessel at room temperature over a period of time of 10 minutes, under stirring. The solid was then immediately dissolved in the medium upon addition. The NaOH titration shows that the conversion of NH₄⁺ ions is superior to 90%. No water was generated. The medium was concentrated under reduced pressure (P=20 mbar, T=0° C.). 120 ml of TFE was then added and the concentration repeated under the same conditions a second time. 13 g of a viscous transparent liquid was obtained. ¹⁹F NMR is consistent with LiFSI, NH₄FSI and TFE mixture. No other fluorinated species are detected. The NaOH titration suggests a high conversion into LiFSI.

1. A process for manufacturing a bis(fluorosulfonyl)imide (MFSI) salt of formula (I):
[F—SO₂—N⁻—SO₂—F] M⁺ (I)
wherein
M is selected from the group consisting of Li, Na, K, Rb, Cs and Fr, and said process comprises:
reacting an ammonium salt of bis(fluorosulfonyl)imide (NH₄FSI) of formula (II):
[F—SO₂—N⁻—SO₂—F] NH₄⁺ (II)
with a fluorine-containing alkoxide of formula (III) or (III*):
RO⁻M⁺ (III)
[RO⁻M⁺][n ROH] (III*)
wherein
each R is independently selected from the group consisting of C₁-C₂₀ fluoroalkyl, C₁-C₂₀ fluoroalkenyl and C₁-C₂₀ fluoroalkynyl,.
M is selected from the group consisting of Li, Na, K, Rb, Cs and Fr,
n is an integer ranging from 1 to 10, and
NH₄FSI salt is optionally in a form of a solvate comprising:
50 to 99.9 wt. %, of the NH₄FSI salt, and
0.1 to 50 wt. %, of at least one solvent S₂.

2. The process according to claim 1, wherein in the fluorine-containing alkoxide (III) or (III*), each R independently complies with formula R_{F}-L, in which:
R_{F} is selected from the group consisting of C₁-C₃ perfluoroalkyl, C₁-C₃ perfluoroalkenyl and C₁-C₃ perfluoroalkynyl, and
L is selected from the group consisting of C₁-C₃ alkylene, C₁-C₃ alkenylene and C₁-C₃ alkynylene.

3. The process according to claim 2, wherein in the fluorine-containing alkoxide (III) or (III*), each R independently complies with formula R_{F}-L in which:
R_{F} is a C₁-C₂ perfluoroalkyl, and
L is a C₁-C₂ alkylene.

4. The process according to claim 3, wherein in the fluorine-containing alkoxide (III) each R independendly complies with formula R_{F}-L in which:
R_{F} is CF₃, and
L is CH₂.

5. The process according to claim 1, comprising reacting NH₄FSI with a fluorine-containing alkoxide of formula (IIIa):
[CF₃CH₂O⁻Li⁺] [2 CF₃CH₂OH] (IIIa)
to obtain a lithium salt of bis(fluorosulfonyl)imide (LiFSI).

6. The process according to claim 1, wherein the reaction is performed in a solvent complying with formula ROH, in which R is selected from the group consisting of C₁-C₂₀ fluoroalkyl, C₁-C₂₀ fluoroalkenyl and C₁-C₂₀ fluoroalkynyl.

7. The process according to claim 1, wherein the reaction is performed at a temperature between 0 and 200° C.

8. The process according to claim 1, wherein the reaction is performed at a pressure varying between 0.01 and 1 atm.

9. The process according to claim 1, comprising a step of solubilizing NH₄FSI in a solvent, before the step of reacting NH₄FSI with the alkoxide (III) or (III*).

10. The process according to claim 1, comprising a step of concentrating MFSI under reduced pressure, after the step of reacting NH₄FSI with the alkoxide (III) or (III*).

11. The process according to claim 1, comprising a step of preparation of NH₄FSI (II), before the step of reacting NH₄FSI (II) with the alkoxide (III) or (III*), by:
reacting an ammonium salt of bis(chlorosulfonyl)imide (NH₄CSI) with a fluorinating agent, or
reacting a bis(chlorosulfonyl)imide (HCSI) with a fluorinating agent of formula NH₄F (HF) p, wherein p represents a number between 0 to 10.

12. The alkali metal salt of bis(fluorosulfonyl)imide (MFSI) obtained from the process of claim 1.

13. (canceled)

14. The process according to claim 1, wherein said fluorine-containing alkoxide of formula (III*) complies with formula (IIIa):
[CF₃CH₂O⁻Li⁺] [2 CF₃CH₂OH] (IIIa)
and the bis(fluoro sulfonyl)imide salt (MFSI) is a lithium salt complying with formula (Ia):
[F—SO₂—N⁻—SO₂—F] Li⁺ (Ia).

15. A process for preparing a fluorine-containing alkoxide of formula (III) or (III*):
RO⁻M⁺ (III)
[RO⁻M⁺] [n ROH] (III*)
wherein
each R is independently selected from the group consisting of C₁-C₂₀ fluoroalkyl, C₁-C₂₀ fluoroalkenyl and C₁-C₂₀ fluoroalkynyl, and
M represents an alkali metal, and
n is an integer ranging from 1 to 10,
said process comprising the step of reacting an alcohol of formula (IV):
ROH (IV)
with an alkali hydroxide of formula (V), or an a hydrate thereof:
MOH (V).

16. The process of claim 1, wherein the at least one solvent S₂ is selected from the group consisting of cyclic and acyclic ethers.

17. The process of claim 15, wherein the alkali metal is selected from the group consisting of Li, Na, K, Rb, Cs and Fr.
